(19) United States
(12) Patent Application Publication
(10) Pub. No.: US 2024/0084250 A1
(43) Pub. Date: Mar. 14, 2024

(54) METHODS AND DEVICES FOR SEPARATION OF MOTILE SPERM

(71) Applicant: NATIONAL UNIVERSITY OF SINGAPORE, Singapore (SG)

(72) Inventors: Narjes ALLAHRABBI, Toronto (CA); Lih Feng CHEOW, Singapore (SG); Xu CUI, Singapore (SG); Megan SOO, Singapore (SG)

(73) Assignee: NATIONAL UNIVERSITY OF SINGAPORE, Singapore (SG)

(21) Appl. No.: 18/272,768

(22) PCT Filed: Jan. 18, 2022
(86) PCT No.: PCT/SG2022/050015

§ 371 Date: Jul. 17, 2023

(30) Foreign Application Priority Data
Jan. 18, 2021 (SG) 10202100509W

(51) Int. Cl. C12N 5/076; C12M 1/00; C12M 1/26; C12M 1/34; C12M 3/06; C12N 5/071

(52) U.S. Cl. CPC C12N 5/061; C12M 23/16; C12M 33/04; C12M 41/46; C12M 47/04; C12N 5/0612

(57) ABSTRACT

The present disclosure provides methods and devices for separation of motile sperm. A method of separating motile sperm comprises: introducing a fluid sample comprising motile sperm to an inlet portion of a microfluidic device; and causing the fluid sample to flow through a separation portion of the microfluidic device at a flow velocity within a rheotaxis range such that motile sperm in the sample undergo rheotaxis and remain in the separation whereas a part of the fluid sample flows out of the separation zone through an outlet portion of the microfluidic device.

### TECHNICAL FIELD

The present disclosure relates to methods of processing sperm and in particular to methods and devices for separation of motile sperm.

### BACKGROUND

Approximately 50% of fertility issues are attributable to men, therefore an important aspect of infertility treatment relates to processing sperm. Intrauterine Insemination (IUI), a relatively non-invasive method where sperms are washed, concentrated, and placed directly into the uterus, can often increase the chances of pregnancy in the cases of low sperm count or low sperm motility. Two problems with the IUI procedure today are limited accessibility and low sample quality due to reactive ion species (ROS) generated during centrifugation step. Therefore, there is a demand to decrease cost and trouble of IUI via less IUI cycles and yet increase its success rate by including as much motile sperm as possible.

To date, very few microfluidic sperm preparation devices have the capability to process physiologically relevant volumes of semen for clinical usage in IUI. Another restriction of reported microfluidic sperm preparation devices is the limited number of isolated motile sperm as they separate only progressively motile sperms. Low sample retrieval is detrimental to patients with low sperm count, as sperm count in excess of 10 million is recommended for an effective IUI procedure. Studies have shown that the sperms with minimal motility still can result in successful pregnancy (Chen, H., et al. (2017). “A successful pregnancy using completely immotile but viable frozen-thawed spermatozoa selected by laser.” Clin Exp Reprod Med 44(1): 52-55). Therefore, most existing techniques are only suitable for in vitro fertilization (IVF) and intracytoplasmic sperm injection (ICSI) that require much less sperm count.

Examples of sperm separation and sorting are provided in the following documents:
Wu, J. K., et al. (2017). “High-throughput flowing upstream sperm sorting in a retarding flow field for human semen analysis.” Analyst 142(6): 938-944.
Hwang, B., et al. (2019). “Rheotaxis Based High-Throughput Motile Sperm Sorting Device.” International Journal of Precision Engineering and Manufacturing 20(6): 1037-1045.
Zaferani, M., et al. (2018). “Rheotaxis-based separation of sperm with progressive motility using a microfluidic corral system.” Proceedings of the National Academy of Sciences of the United States of America 115(33): 8272-8277.

### SUMMARY

The present disclosure provides a point-of-care sperm preparation microfluidic device that relies on sperm's rheotaxis behavior to concentrate motile sperm cells with various ranges of motilities and passively improve the quality of sperms, without the need to bulky lab instruments such as mechanical centrifugation. The obtained sperm concentration satisfies the effective IUI guidelines (>10 million).

According to a first aspect of the present disclosure, a method of separating motile sperm is provided. The method comprises: introducing a fluid sample (diluted semen sample) comprising motile sperm to an inlet portion of a microfluidic device; and causing the fluid sample to flow through a separation portion of the microfluidic device at a flow velocity within a rheotaxis range such that motile sperm in the sample undergo rheotaxis and remain in the separation whereas a part of the fluid sample flows out of the separation zone through an outlet portion of the microfluidic device.

The method may further comprise extracting the motile sperm from the separation portion of the microfluidic device by causing fluid flow through the separation portion at a flow velocity above the rheotaxis range.

The rheotaxis range is a flow velocity range of 17 to 100 μm/s.

In some embodiments, the method further comprises introducing a buffer fluid to the inlet portion of the microfluidic device.

The fluid sample is caused to flow through the separation portion of the microfluidic device creating a fluid pressure difference between the inlet portion and the outlet portion of the microfluidic device.

The fluid pressure difference may be created by a height difference between a fluid level of an inlet reservoir coupled to the inlet portion of the microfluidic device and a fluid level of an outlet reservoir coupled to the outlet portion of the microfluidic device.

The fluid pressure difference may be created by a syringe coupled to the inlet portion of the microfluidic device and/or a syringe coupled to the outlet portion of the microfluidic device.

According to a second aspect of the present disclosure, a microfluidic device for separating motile sperm is provided. The microfluidic device comprises: an inlet portion configured to receive a fluid sample comprising motile sperm; a separation portion coupled to the inlet portion; and an outlet portion coupled to the separation portion, wherein the separation portion is configured such that, when a separation pressure difference is applied between the inlet portion and the outlet portion, fluid flow in the separation portion takes place at a flow velocity within a rheotaxis range such that motile sperm in the sample undergo rheotaxis remain in the separation portion whereas a part of the fluid sample flows out of the separation zone through the outlet portion.

The inlet portion and the outlet portion may be configured such that when the separation pressure difference is applied between the inlet portion and the outlet portion, fluid flow in the inlet portion and the outlet portion takes place at a flow velocity above the rheotaxis range.

The rheotaxis range is a flow velocity range of 17 to 100 μm/s.

The microfluidic device may further comprise an inlet reservoir coupled to the inlet portion and configured to hold the fluid sample and an outlet reservoir coupled to the outlet portion.

In an embodiment, the inlet reservoir has a smaller cross-sectional area than the outlet reservoir. This results in the fluid level of the outlet reservoir having a smaller dependency on the flow of fluid through the device than the fluid level of the inlet reservoir.

A plurality of pillars may be provided in the separation portion to support the top of the chamber forming the separation portion.

The inlet portion and/or the outlet portion may comprise a branching channel. This provides for even flow across different parts or different channels of the separation portion.

In an embodiment the separation portion comprises a plurality of separate channels. The channels may have a diameter that varies along a length thereof to provide fast flow zones and slow flow zones.

In an embodiment the separation portion comprises a single chamber.

A syringe coupled to the inlet portion or the outlet portion may be provided to provide a pressure difference.

### BRIEF DESCRIPTION OF THE DRAWINGS

In the following, embodiments of the present invention will be described as non-limiting examples with reference to the accompanying drawings in which:

FIG. 1A shows sperm and other cells in a semen sample;

FIG. 1B shows motile sperm undergoing rheotaxis in when subjected to a fluid flow;

FIG. 2A to FIG. 2D show a method of separating motile sperm using a microfluidic device according to an embodiment of the present invention;

FIG. 3A shows a top view of a microfluidic device according to an embodiment of the present invention;

FIG. 3B shows a top view of a microfluidic device according to an embodiment of the present invention;

FIG. 4A shows a cross section of a microfluidic device according to an embodiment of the present invention;

FIG. 4B shows a cross section of a microfluidic device in which a pressure difference between the inlet and outlet is provided by a syringe at the inlet according to an embodiment of the present invention;

FIG. 4C shows a cross section of a microfluidic device in which a pressure difference between the inlet and outlet is provided by a syringe at the outlet according to an embodiment of the present invention;

FIG. 5A shows a channel design for a microfluidic device according to an embodiment of the present invention; and

FIG. 5B shows a microfluidic device according to an embodiment of the present invention and microscope images at different locations.

### DETAILED DESCRIPTION

The present disclosure relates to methods and devices for sperm preparation which are based on rheotaxis. Rheotaxis is a type of movement where an organism turns to face an oncoming current and swims upstream or holds their position by swimming against the current. Healthy, motile sperm cells have been found to undergo rheotaxis in the flow velocity range of 17 to 100 μm/s.

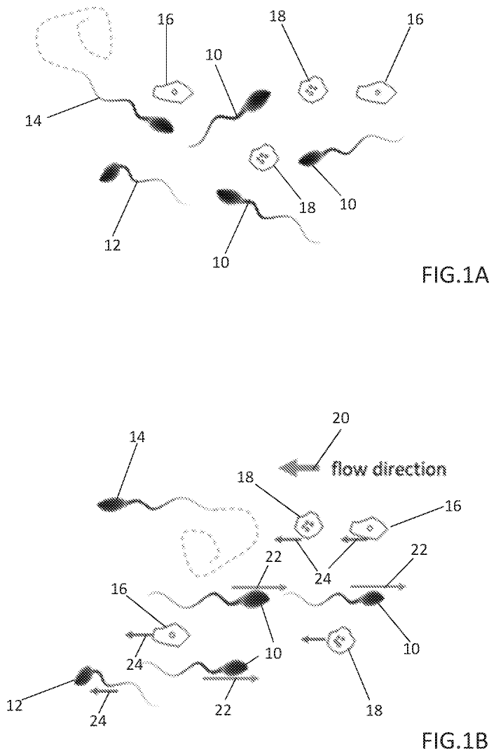

FIG. 1A shows sperm and other cells in a semen sample. As shown in FIG. 1A, the semen sample comprises motile sperm 10 which are able to swim in all directions. The sample also comprises non-motile sperm 12 and non-progressively motile sperm 14 which are either incapable of swimming or are limited in the directions in which they can swim. The semen sample further comprises non-sperm cells including white blood cells 16 and epithelial cells 18. The sperm and other cells are suspended in a fluid. As shown in FIG. 1A, when there is no fluid flow, the motile sperm 10 swim in all directions.

FIG. 1B shows motile sperm undergoing rheotaxis in when subjected to a fluid flow. In the presence of an external fluid flow 20, the head of the motile sperm 10 orients in a direction 22 against the flow as a result of the change in physical forces. The motile sperm 10 thus reorient themselves and swim against the fluid flow 20. This behaviour, is called rheotaxis, and has also been observed in vivo. Motile sperm move against the vaginal discharge to concentrate at folds in fallopian tubes, maximizing their chances to fertilize eggs.

Non-motile sperm 12, white blood cells 16, and epithelial cells 18 do not undergo rheotaxis and therefore move in the direction 24 of the fluid flow. Thus, the process of rheotaxis can be used to separate the motile sperm 10 from non-motile sperm and other cells.

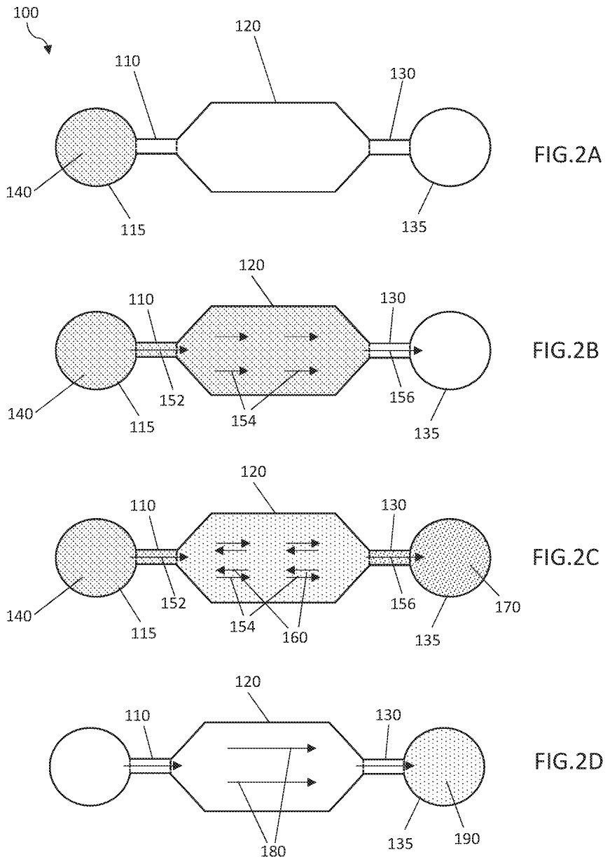

A method of separating motile sperm using a microfluidic device according to an embodiment of the present invention will now be described with reference to FIG. 2A to FIG. 2D.

FIG. 2A shows a microfluidic device according to an embodiment of the present invention. The microfluidic device 100 comprises an inlet portion 110, a separation portion 120 and an outlet portion 130. The inlet portion 110 is coupled to an inlet side of the separation portion 120 and the outlet portion 130 is coupled to an outlet side of the separation portion 120. An inlet reservoir 115 is coupled to the inlet portion 110. An outlet reservoir 135 is coupled to the outlet portion 130. As shown in FIG. 2A, the separation portion 120 is wider than the inlet portion 110, and the separation portion 120 is also wider than the outlet portion 130. This means that when fluid flows from the inlet portion 110 to the outlet portion 130 through the separation portion 120, the fluid flow velocity through the inlet portion 110 will be greater than the fluid flow velocity in the separation portion 120. Similarly, the fluid flow velocity through the outlet portion 130 will be greater than the fluid flow velocity in the separation portion 120.

The channels forming the inlet portion 110, the separation portion 120, and the outlet portion 130 may have a height in the range of 10 μm to 1000 μm and a width in the range of 10 μm to 20 cm.

As shown in FIG. 2A, a fluid sample 140 comprising motile sperm is introduced into the inlet reservoir 115. The fluid sample 140 may comprise non-motile sperm and other cells as shown in FIG. 1A.

As shown in FIG. 2B, a pressure difference is applied between the inlet portion 110 and the outlet portion 130. This pressure difference causes a fluid flow 152 through the inlet portion 110, a fluid flow 154 through the separation portion 120 and a fluid flow 156 through the outlet portion 130. As described above, the separation portion 120 is wider than the inlet portion 110, and the separation portion 120 is also wider than the outlet portion 130. This means that the fluid flow 152 though the inlet portion has a flow velocity which is greater than that of the fluid flow 154 through the separation portion 120. Similarly, the fluid flow 156 through the outlet portion 156 has a greater flow velocity than that of the fluid flow 154 through the separation portion 120.

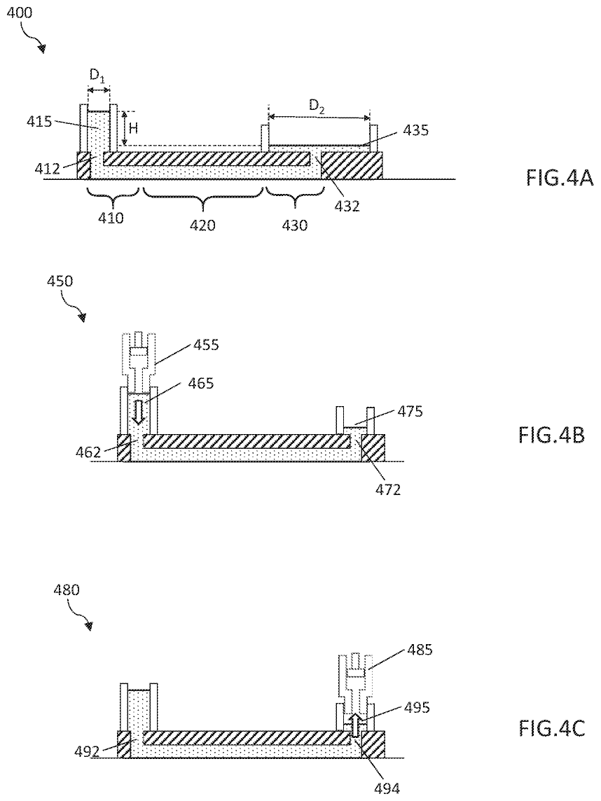

As is described in more detail below with reference to FIG. 4A to FIG. 4C, the pressure difference between the inlet portion 110 and the outlet portion 130 may be applied by a difference in fluid level between the inlet reservoir 115 and the outlet reservoir 135. Alternatively, the pressure difference can be applied by a syringe coupled to the inlet reservoir and/or a syringe coupled to the outlet reservoir 135.

The pressure difference applied between the inlet portion 110 and the outlet portion 130 is referred to as a separation pressure difference when it creates a flow velocity 154 in the separation portion 120 at which motile sperm undergo rheotaxis. This situation is shown in FIG. 2C.

As shown in FIG. 2C, the separation pressure difference causes the fluid sample 140 to flow from the inlet reservoir 115 through the inlet portion 110 into the separation portion 120. Because the flow velocity of this fluid flow 152 is above the range of flow velocities at which motile sperm undergo rheotaxis, motile sperm in the fluid sample 140 along with the other constituents of the fluid sample 140 are carried into the separation portion 120. Because the separation portion 120 is wider than the inlet portion 110, the flow velocity of the fluid flow 154 in the separation portion 120 is within the range of flow velocities at which motile sperm undergo rheotaxis. As shown in FIG. 2C, since the motile sperm in the fluid sample 140 undergo rheotaxis, they swim in a direction 160 opposite to the fluid flow 154 or hold their position in the separation portion 120. This results in the motile sperm remaining in the separation portion 120. Non-motile sperm and other cells in the fluid sample are carried out of the separation portion 120 by the fluid flow 156 through the outlet portion 130 and into the outlet reservoir 135. Thus, non-motile sperm, and other cells are collected as waste 170 in the outlet reservoir 135.

The process shown in FIG. 2C is continued until a sufficient amount of the fluid sample 140 has flowed through the inlet portion 110 into the separation portion 120. In some embodiments, a buffer fluid may be added to the inlet reservoir 115 during the process to maintain the fluid level in the inlet reservoir 115 at a sufficient level to provide the separation pressure difference between the inlet portion 110 and the outlet portion 130.

When the process shown in FIG. 2C is completed, the motile sperm (i.e. progressively motile and non-progressively motile) will remain in the separation portion 120 whereas the non-motile sperm, and other cells will be collected as waste 170 in the outlet reservoir 135. At this stage, the waste 170 in the outlet reservoir 135 is removed and discarded, or alternatively, a new, empty outlet reservoir 135 may be connected to collect the motile sperm from the separation portion 120. Then, the motile sperm are collected as shown in FIG. 2D.

FIG. 2D shows the process of collecting motile sperm from the separation portion 120. A collection pressure difference is applied between the inlet portion 110 and the outlet portion 130. As shown in FIG. 2D, the collection pressure difference causes a fluid flow 180 through the separation portion 120 with a flow velocity above the range of velocities at which motile sperm undergo rheotaxis. This results in the motile sperm remaining in the separation portion 120 after the separation process shown in FIG. 2C being carried through the outlet portion 130 by the fluid flow. Thus, motile sperm 190 are collected in the outlet reservoir 135. The collection pressure difference may be applied by adding buffer fluid or sperm washing media to the inlet reservoir 115 to provide an increased fluid level difference between the inlet reservoir and the outlet reservoir and thereby increase the pressure difference between the inlet portion 110 and the outlet portion 130. Alternatively, the collection pressure difference can be applied by one or more syringes.

As described above, to select and enrich motile sperm cells, diluted semen fluid sample is injected through the microfluidic device at a moderate flow rate (10-50 μL/min) to retain only the motile sperm cells in the separation portion. Subsequently, injection of sperm washing media through the device at high flow rate (500 μL/min) can elute the concentrated motile sperm cells for direct use in IUI. Thus, a 500 μL of semen sample could be processed in 10 minutes.

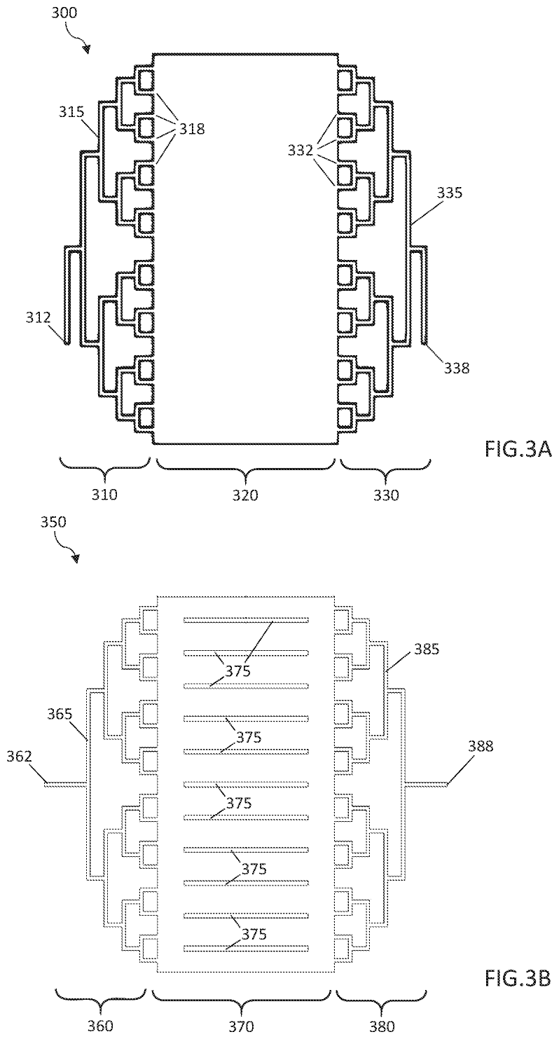

FIG. 3A shows a top view of a microfluidic device according to an embodiment of the present invention. As shown in FIG. 3A, the microfluidic device 300 comprises an inlet portion 310 which is formed from a set of branching inlet channels 315, a separation portion 320 which is a rectangular chamber and an outlet portion 330 which is formed form a set of branching outlet channels 335.

The inlet channels 315 start from an inlet 312 which is coupled to the inlet reservoir. The inlet channels 315 branch into two four times so that there are 16 inlet channel openings 318 distributed over an inlet side of the separation portion 320. The outlet channels 335 run from 16 outlet channel openings 332 which are distributed over an outlet side of the separation portion 320. The inlet side of the separation portion 320 is opposite the outlet side of the separation portion 320 and as shown in FIG. 3A, the inlet side and the outlet side of the separation portion 320 approximately twice as long as the separation between them. The outlet channels 335 branch four times such that the outlet channels 335 combine to connect to a single outlet 338 which is coupled to the outlet reservoir.

An embodiment of the microfluidic device 300 shown in FIG. 3A has the following dimensions: separation portion length (distance between inlet side and outlet side) 41 mm; separation portion breadth (length of inlet side or outlet side): 82 mm; width of channels 1 mm; height of channels and chamber of separation portion 0.2 mm. The microfluidic device 300 may be formed from polydimethylsiloxane (PDMS), and in some embodiments, the microfluidic device may be formed on a glass slide with the channels of the inlet portion and outlet portion and the chamber of the separation portion formed from PDMS.

As shown in FIG. 3A, the inlet and outlet channels were branched four times to result in 16 openings into the central chamber. As the parallel channels are designed to be the same length and height, the hydrodynamic resistance in the branches at each junction are identical. The evenly branched channels encourage uniform flow velocity into the central chamber. Hydrodynamic resistance can be calculated with the following equations:

$${R}_{h} = \frac{12 μL}{W \times {H}^{3}}$$

Fluidic viscosity, μ: 10⁻³ Pa·s; Channel width, W; Channel height, H

For channels in parallel:

$$\frac{1}{{R}_{h}} = \frac{1}{{R}_{h 1}} + \frac{1}{{R}_{h 2}} + …$$

As discussed in more detail below, if the hydrodynamic resistance is known, the necessary pressure differences between the inlet and the outlet can be determined to provide flow velocities in the separation portion that are within the range at which motile sperm undergo rheotaxis.

FIG. 3B shows a top view of a microfluidic device according to an embodiment of the present invention. The design of the microfluidic device 350 shown in FIG. 3B includes pillars provided in the separation portion to support the top of the chamber of the separation portion.

As shown in FIG. 3B, the microfluidic device 350 comprises an inlet portion 360 which is formed from a set of branching inlet channels 365, a separation portion 370 which is a rectangular chamber and an outlet portion 380 which is formed form a set of branching outlet channels 385.

An inlet 362 is coupled to the inlet channels 365. It is noted that the position of the inlet 362 shown in FIG. 3B differs from the position of the inlet 312 shown in FIG. 3A. The reason for this difference is to accommodate a different length for the inlet channels. The length of the inlet channels 365 and the length of the outlet channels 385 is determined to provide a required flow resistance and the positioning of the inlet 362 and the outlet 388 is modified to provide for the required channel lengths. The outlet 388 from the outlet channels 385 has a corresponding position to that of the inlet 362. The general arrangement of the inlet channels 365 and their connection to the inlet side of the separation portion is as described above with reference to FIG. 3A.

A plurality of rectangular pillars 375 are provided within the chamber formed by the separation portion 370. The pillars 375 are orientated parallel to the flow direction through the chamber of the separation portion and are spaced at equal intervals. The pillars 375 extend from close to the inlet side of the separation portion 370 to close to the outlet side of the separation portion 370. The pillars 375 are provided to support the top of the separation portion 370 and to prevent the top from sagging into the chamber of the separation portion 370 and impeding fluid flow through the separation portion 370.

An embodiment of the microfluidic device 350 shown in FIG. 3B has the following dimensions: separation portion length (distance between inlet side and outlet side) 40 mm; separation portion breadth (length of inlet side or outlet side): 80 mm; dimensions of pillars in separation portion: 1 mm×28 mm; width of channels 0.8 mm; height of channels and chamber of separation portion 0.2 mm.

The microfluidic device may use gravitational force to create a pressure difference, ΔP, across the device between the inlet and the outlet. By adjusting the dimensions of the channels and chamber of the device, the hydrodynamic resistance can be changed accordingly. Flow rate is inversely proportional to resistance by the formula Q=ΔP/R; it is also related to flow velocity by the formula Q=A_{Cross Section}×V_{Average}. Hence, the area, resistance, and velocity are related by:

$${A}_{Cross Section} \times R = \frac{Δ P}{{V}_{Average}}$$

where ΔP is a fixed value, determined by the sample volume and reservoir diameter. With that, theoretical calculations and simulations were conducted to optimise the device dimensions in order to change the hydrodynamic resistance and consequently achieve the optimal flow velocity range for sperm rheotaxis.

FIG. 4A shows a cross section of a microfluidic device according to an embodiment of the present invention. As shown in FIG. 4A, the microfluidic device 400 comprises an inlet portion 410, a separation portion 420 and an outlet portion 430. An inlet 412 couples the inlet portion 410 to an inlet reservoir 415 and an outlet 432 couples the outlet portion 430 to an outlet reservoir 435.

In the microfluidic device 400 shown in FIG. 4A, the pressure difference between the inlet 412 and the outlet 432 is created by the height difference H between the fluid level in the inlet reservoir 415 and the fluid level in the outlet reservoir 435. Thus, in this embodiment, gravitational forces are used to create a pressure difference and induce fluid flow from the inlet 412 to the outlet 432. Based on the dimensions described above with reference to FIG. 3A and FIG. 3B, it is calculated that a height difference H of approximately 0.8 cm is required to provide the scenario described above with reference to FIG. 2C, namely a situation in which the flow velocity in the separation portion 420 is within the range for motile sperm to undergo rheotaxis, whereas the flow velocity in the inlet portion 410 and the outlet portion 430 is above the range for motile sperm to undergo rheotaxis.

As shown in FIG. 4A, the diameter D₁ of the inlet reservoir 415 is smaller than the diameter D₂ of the outlet reservoir 435. This means that as the fluid sample flows through the microfluidic device 400, the increase in the fluid level of the outlet level will be relatively small. Therefore, the fluid level of the inlet reservoir 415 can be topped up with a buffer fluid during the processing to maintain the height difference without being greatly affected by any increase in the fluid level in the outlet reservoir.

In order to collect the motile sperm accumulated in the separation portion 420, the fluid level in the inlet reservoir can be increased to provide a greater pressure difference and therefore a higher flow velocity through the separation region 420.

It will be appreciated that while the above description with reference to FIG. 4A discusses the relative diameters D₁ and D₂ of the inlet reservoir 415 and the outlet reservoir 435, it is the cross-sectional area of the respective reservoirs that is the important factor and depending on the configuration of the reservoirs on the microfluidic device 400 the shapes of the reservoirs may be different.

As described above with reference to FIG. 4A, the pressure difference between the inlet and the outlet of the microfluidic device may be provided by gravitational force due to the difference in height of fluid levels in the inlet and outlet reservoirs. Alternatively, the pressure difference may be provided by a syringe at the inlet and/or a syringe at the outlet. This is described below with reference to FIG. 4B and FIG. 4C.

FIG. 4B shows a cross section of a microfluidic device in which a pressure difference between the inlet and outlet is provided by a syringe at the inlet according to an embodiment of the present invention.

As shown in FIG. 4B, a syringe 455 is applied over the inlet reservoir 465 of the microfluidic device 450. Downward actuation of the syringe 455 applies a pressure at the inlet 462 of the microfluidic device 450. This creates a pressure difference between the inlet 462 and the outlet 472 which in turn results in fluid flow from the inlet reservoir 465 to the outlet reservoir.

FIG. 4C shows a cross section of a microfluidic device in which a pressure difference between the inlet and outlet is provided by a syringe at the outlet according to an embodiment of the present invention.

As shown in FIG. 4C, a syringe 485 is applied over the outlet reservoir 495 of the microfluidic device 480. Upward actuation of the syringe 485 creates a negative pressure at the outlet reservoir 495 which creates in a pressure difference between the inlet 492 and the outlet 494 and results in fluid flow through the microfluidic device 480.

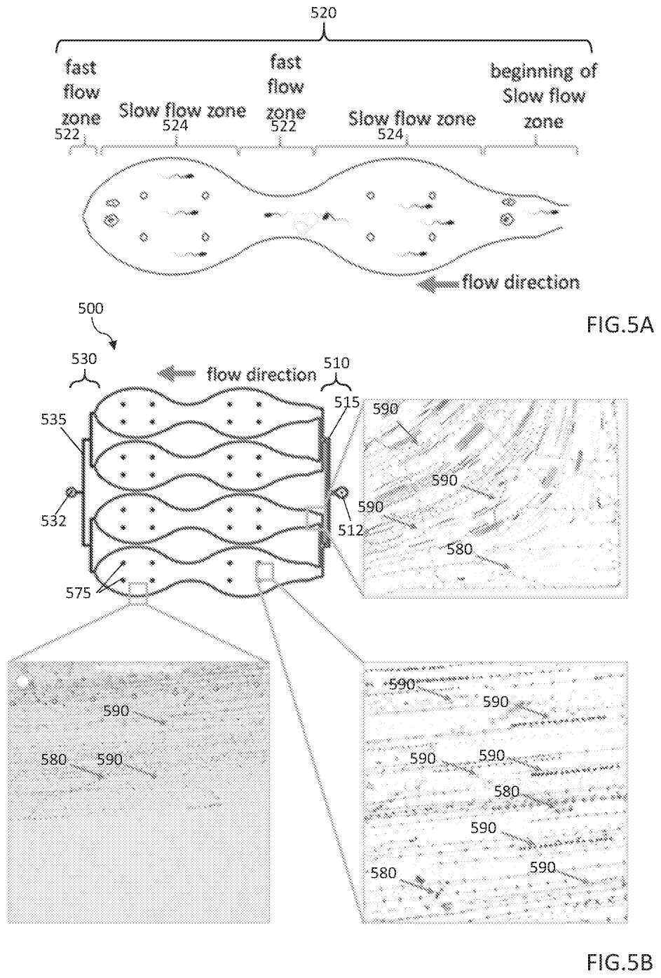

FIG. 5A shows a channel design for a microfluidic device according to an embodiment of the present invention. As shown in FIG. 5A, the microfluidic device has a separation portion 520 formed from a channel with undulating sides such that there is a sequence of slow flow zones 624 and fast flow zones 522. The fast flow zones 522 are formed by narrow portions of the channel and the slow flow zones are formed by wide portions of the channel. A wavy microfluidic channel design as shown in FIG. 5A establishes of a velocity gradient within the microfluidic device which allows trapping of motile sperm with different forward velocities.

FIG. 5B shows a microfluidic device according to an embodiment of the present invention and microscope images at different locations.

As shown in FIG. 5B, the microfluidic device 500 is formed from four channels as described above with reference to FIG. 5A. The four channels are connected in parallel. An inlet portion 510 formed by branching inlet channels connects an inlet 512 to each of the four channels. An outlet portion 530 formed by branching outlet channels 535 connects each of the four channels to an outlet 532. A plurality of pillars 575 are arranged in the slow flow zones 524 to support the top of the channels.

FIG. 5B shows miniaturized view of the microchannel with snapshots of the beginning of microchannel, the wall region and the middle of the microchannel expansion of zone. The path for sperm swimming was recorded by microscope and it is shown by superimposing the frames of microscope image. All the arrows show sperms swimming upstream while other sperms are moving downstream with flow. The arrows labelled 580 show sperms regaining control of its path. The sperm initially flow along with the stream (right to left) and then makes a U-turn to align opposite the flow stream. If the sperm head moves toward the region of low velocity, the movement can be sustained. The arrows labeled 590 orange arrows show the sperms under rheotaxis following the streamlines.

Parallel microfluidic channels with large channel heights will enable physiologically relevant amounts of semen to be processed in a short time. To select and enrich for motile sperm cells, diluted semen sample is injected through the device as a moderate flow rate (10 μL/min) to retain only the motile sperm cells in the expansion region. Subsequently, injection of sperm washing media through the device at high flow rate (500 μL/m in) can elute the concentrated motile sperm cells for direct use in IUI.

The devices and methods described above allow a high throughput: 500 μl of semen sample can be processed in 10 minutes. The development of a passive device is relevant in reducing resources required for manufacturing and simplifying operations such that minimal training is needed to use the device.

Whilst the foregoing description has described exemplary embodiments, it will be understood by those skilled in the art that many variations of the embodiments can be made within the scope and spirit of the present invention.

1. A method of separating motile sperm, the method comprising:
introducing a fluid sample comprising motile sperm to an inlet portion of a microfluidic device; and
causing the fluid sample to flow through a separation portion of the microfluidic device at a flow velocity within a rheotaxis range such that motile sperm in the sample undergo rheotaxis and remain in the separation whereas a part of the fluid sample flows out of the separation zone through an outlet portion of the microfluidic device.

2. A method according to claim 1, further comprising extracting the motile sperm from the separation portion of the microfluidic device by causing fluid flow through the separation portion at a flow velocity above the rheotaxis range.

3. A method according to claim 1, wherein the rheotaxis range is a flow velocity range of 17 to 100 μm/s.

4. A method according to claim 1, further comprising introducing a buffer fluid to the inlet portion of the microfluidic device.

5. A method according to claim 1, wherein causing the fluid sample to flow through the separation portion of the microfluidic device comprises creating a fluid pressure difference between the inlet portion and the outlet portion of the microfluidic device.

6. A method according to claim 5, wherein the fluid pressure difference between the inlet portion and the outlet portion of the microfluidic device is created by a height difference between a fluid level of an inlet reservoir coupled to the inlet portion of the microfluidic device and a fluid level of an outlet reservoir coupled to the outlet portion of the microfluidic device.

7. A method according to claim 5, wherein the fluid pressure difference between the inlet portion and the outlet portion of the microfluidic device is created by a syringe coupled to the inlet portion of the microfluidic device and/or a syringe coupled to the outlet portion of the microfluidic device.

8. A microfluidic device for separating motile sperm, the microfluidic device comprising:
an inlet portion configured to receive a fluid sample comprising motile sperm;
a separation portion coupled to the inlet portion; and
an outlet portion coupled to the separation portion,
wherein the separation portion is configured such that, when a separation pressure difference is applied between the inlet portion and the outlet portion, fluid flow in the separation portion takes place at a flow velocity within a rheotaxis range such that motile sperm in the sample undergo rheotaxis remain in the separation portion whereas a part of the fluid sample flows out of the separation zone through the outlet portion.

9. A microfluidic device according to claim 8, wherein the inlet portion and the outlet portion are configured such that when the separation pressure difference is applied between the inlet portion and the outlet portion, fluid flow in the inlet portion and the outlet portion takes place at a flow velocity above the rheotaxis range.

10. A microfluidic device according to claim 8, wherein the rheotaxis range is a flow velocity range of 17 to 100 μm/s.

11. A microfluidic device according to claim 8, further comprising an inlet reservoir coupled to the inlet portion and configured to hold the fluid sample and an outlet reservoir coupled to the outlet portion.

12. A microfluidic device according to claim 11, wherein the inlet reservoir has a smaller cross-sectional area than the outlet reservoir.

13. A microfluidic device according to claim 8, wherein a plurality of pillars are provided in the separation portion.

14. A microfluidic device according to claim 8, wherein the inlet portion and or the outlet portion comprises a branching channel.

15. A microfluidic device according to claim 8, wherein the separation portion comprises a plurality of separate channels.

16. A microfluidic device according to claim 15, wherein each of the separate channels has a diameter that varies along a length thereof to provide fast flow zones and slow flow zones.

17. A microfluidic device according to claim 8, wherein the separation portion comprises a single chamber.

18. A microfluidic device according to claim 8, further comprising a syringe coupled to the inlet portion or the outlet portion.
